(19) United States
(12) Patent Application Publication
(10) Pub. No.: US 2022/0395224 A1
(43) Pub. Date: Dec. 15, 2022

(54) URINE COLOR SYSTEM AND METHOD FOR USE OF THE SAME

(71) Applicants: Floris Wardenaar, Chandler, AZ (US); Dean Bacalzo, Broomfield, CO (US)

(72) Inventors: Floris Wardenaar, Chandler, AZ (US); Dean Bacalzo, Broomfield, CO (US)

(21) Appl. No.: 17/806,697

(22) Filed: Jun. 13, 2022

Related U.S. Application Data
(60) Provisional application No. 63/210,434, filed on Jun. 14, 2021.

(51) Int. Cl. A61B 5/00; A61B 10/00; G01J 3/52

(52) U.S. Cl. CPC A61B 5/4875; A61B 10/007; G01J 3/52

(57) ABSTRACT

A urine color and concentration assessment system includes a housing, a color chart supported by the housing, and a urine collection container supported by the housing and positioned adjacent the color chart. The urine collection container configured to receive a urine sample for comparing a color of the urine sample to a color of the color chart.

### CROSS-REFERENCE TO RELATED APPLICATIONS

This application claims priority to U.S. Provisional Patent Application No. 63/210,434, filed June 14, 2021, the entire contents of which is incorporated by reference herein.

### FIELD OF INVENTION

The present disclosure relates to a system and method for determining a hydration status of an individual.

### SUMMARY

Urine concentration has been suggested as a biomarker to detect underhydration. This condition presents itself when total body water is conserved due to inadequate fluid intake or fluid replacement, resulting in an increase in urine concentration. This can occur without the perception of thirst or a change in plasma osmolality concentration.

In one embodiment, a color chart for determining hydration status from a void of undiluted urine is disclosed. The color chart includes a first color corresponding to a first range of urine specific gravity values, a second color corresponding to a second range of urine specific gravity values, and a third color corresponding to a third range of urine specific gravity values.

In another embodiment, a color chart for determining hydration status from a void of undiluted urine is disclosed. The color chart consists of seven colors, each of the seven colors corresponding to a range of urine specific gravity values.

In another embodiment, a urine color and concentration assessment system includes a housing, a color chart supported by the housing, and a urine collection container supported by the housing and positioned adjacent the color chart. The urine collection container configured to receive a urine sample for comparing a color of the urine sample to a color of the color chart.

In another embodiment, a urine color and concentration assessment system includes a urine collection container configured to receive a void of undiluted urine, a color chart positioned adjacent to the urine collection container for determining hydration status from the void of undiluted urine, and a light source positioned adjacent to one or both of the color chart and the container to illuminate one or both of the color chart and the container.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a urine color chart according to one embodiment.

FIG. 2A illustrates a perspective view of a urine color and concentration assessment system according to one embodiment and including at least a portion of the urine color chart of FIG. 1.

FIG. 2B illustrates a perspective view of a urine color and concentration assessment system according to another embodiment and including at least a portion of the urine color chart of FIG. 1.

FIG. 2C illustrates a cross-sectional view of the urine color and concentration assessment system of FIG. 2B along the line 2C--2C of FIG. 2C.

FIG. 3A illustrates a perspective view of an exemplary urine color scoring system used to test the urine color chart of FIG. 1.

FIG. 3B illustrates another perspective view of the exemplary urine color scoring system used to test the urine color chart of FIG. 1.

FIG. 3C illustrates another perspective view of an exemplary urine color scoring system used to test the urine color chart of FIG. 1.

FIG. 3D illustrates another perspective view of an exemplary urine color scoring system used to test the urine color chart of FIG. 1.

FIG. 3E illustrates another perspective view of an exemplary urine color scoring system used to test the urine color chart of FIG. 1 while in use.

FIG. 3F illustrates a conventional 8-color urine color chart.

FIG. 3G illustrates the urine color chart of FIG. 1.

FIG. 4 illustrates a table showing participant and urine sample characteristics.

FIG. 5 illustrates a table showing urine color scores, urine concentrations, AUC, accuracy of classification, and receiver operating characteristics (ROC)-based urine color cut-off values scored by participants based on urine samples for two different urine color charts.

FIG. 6 illustrates a Bland-Altman plot based on a comparison between the 8-color chart of FIG. 3F and the urine color chart of FIGS. 1 and 3G.

FIG. 7 illustrates a table showing urine color, AUC, accuracy, and cut-off values for each of the four light conditions with or without a urine color scoring box using the urine color chart of FIG. 1.

FIG. 8 illustrates a table showing a comparison between urine color scores based on fresh and frozen urine samples using the urine color chart of FIG. 1 and a urine color scoring box.

FIG. 9 illustrates urine color (Uc) scored at four different light conditions with and without urine color scoring box.

FIG. 10 illustrates a Bland-Altman plot for fresh vs. frozen comparison based on samples scored on the urine color chart of FIG. 1.

### DETAILED DESCRIPTION

Before any independent constructions of the present disclosure are explained in detail, it is to be understood that the present disclosure is not limited in its application to the details of construction and the arrangement of components set forth in the following description or illustrated in the following drawings. The present disclosure is capable of other independent constructions and of being practiced or of being carried out in various ways.

Use of “including” and “comprising” and variations thereof as used herein is meant to encompass the items listed thereafter and equivalents thereof as well as additional items. Use of “consisting of” and variations thereof as used herein is meant to encompass only the items listed thereafter and equivalents thereof.

Disclosed herein is a urine color (Uc) system and a method for standardization of a urine color chart to assess undiluted urine for hydration status. Hydration status includes dehydration as well as hypohydration, the uncompensated loss of body water, in the human body. The urine color system and method are helpful to standardize urine color scoring when determining hydration status. Standardizing this process helps in early detection of underhydration, which can occur without the perception of thirst or a change in urine concentration. The most accurate way to assess urine concentration involves lab-based techniques, but there is a need for a quicker way that can be assessed rapidly in hospitals, nursing homes, community-dwellings, athletic training facilities, sporting events, or at home. This system and method offer an inexpensive, non-technical and immediate way to assess underhydration without the need for a lab facility.

Introduced 25 years ago, Uc correlates with body water deficits and changes in body water. As a clinical measure of urine concentration, and thus hydration status, urine osmolality and urine specific gravity (USG) have higher sensitivity than Uc. However, Uc is an inexpensive, non-invasive, and easily-performed means of assessing hydration status, even for the untrained individual, and therefore it can serve as a simple tool to identify if athletes need to drink more. A conventional 8-color Uc chart or modifications of this chart can be found in many athletic facilities. However, the accuracy of self-reporting Uc based on multi-shade urine color charts needs validation, for example in athletic populations, allowing health professionals to better educate their athletes on the use and interpretation of multiple shade color charts.

Only three studies have described the validity of Uc self-assessment to determine urine concentration. Two reported the accuracy of Uc scoring in a population of adults and a group of children. A recent study described a novel way athletes can score Uc directly from the toilet bowl. Despite differences in methods, these studies reported similar numbers for correctly classifying low vs. high urine concentrations, based on calculated area under the curve (AUC) values, ranging from 0.67-0.78.

There is substantial variation in Uc charts, as they come in many different colors and sizes, so there can be differences in their comparability and therefore their accuracy. Many charts available on the internet were likely derived from the conventional 8-color Uc chart, which was based on the classification and ranking based on the color of a large number of urine samples. The validity of Uc scoring with color charts has been determined when performed by investigators, however, these studies employed different containers, fluid volumes, and lighting conditions, making it difficult to compare outcomes. The idea is that Uc charts' cut-off values indicate which urine sample has a low vs. high urine concentration, but this depends on methods under which Uc is scored. Cut-off values should therefore be used as a sliding scale, not as a one-size-fits- all approach. The Beer-Lambert law states that light absorbance is equal to the product of (1) the concentration of the solution the light passes through it and the material of the container, (2) solution depth, and (3) the absorption coefficient. Thus, perceived color is influenced by the sample container (glass vs. plastic) and the urine cup's diameter. In combination with the color and intensity of light, these factors will affect light absorption, influencing the rater's response. Therefore, studies examining validity of athlete Uc assessment using multiple color charts, while standardizing for confounding factors such as cup size, material, and light intensity, are non-existent.

As there is a lack of insight in the validation of athlete self-reporting Uc to identify urine with a low vs. high concentration, as well as an insufficient understanding of the comparability of Uc charts, it was an objective to determine the accuracy of urine color (Uc) scoring by an athletic population between a conventional 8-color Uc chart and a newly developed 7-color Uc chart. The aims were as follows: (a) to determine differences in scoring Uc for two different charts; and (b) to investigate the diagnostic ability and the optimal Uc cut-off value of the two Uc charts to assess a low vs. high urine concentration, based on a pre-defined USG and urine osmolality cut-off value.

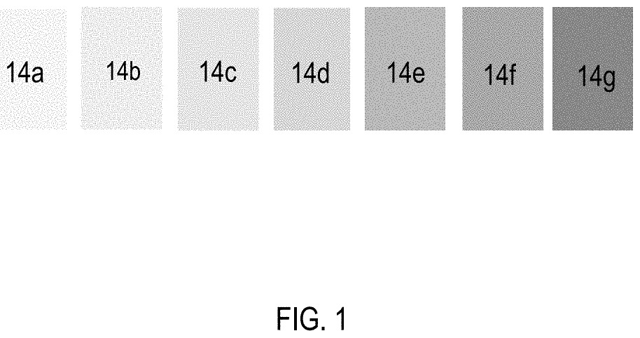

FIG. 1 illustrates a urine color chart 10 for self-assessment of urine concentration. In particular, the color chart 10 may be used for determining hydration status from a void of undiluted urine. The urine color chart 10 includes seven colors 14a-14g with each of the seven colors 14a-14g corresponding to a range of urine specific gravity values. In some embodiments, the ranges of values for urine specific gravity may be non-overlapping. The urine color chart 10 includes a first color 14a corresponding to a first range of urine specific gravity values, a second color 14b corresponding to a second range of urine specific gravity values, a third color 14c corresponding to a third range of urine specific gravity values, a fourth color 14d corresponding to a fourth range for urine specific gravity values, a fifth color 14e corresponding to a fifth range for urine specific gravity values, a sixth color 14f corresponding to a sixth range for urine specific gravity values, and a seventh color 14g corresponding to a sixth range for urine specific gravity values. The first range of specific gravity values include values that are less than 1.017 (inclusive). The second range of specific gravity values include values that are between 1.017 and 1.021 (inclusive). The third range of specific gravity values include values that are between 1.022 and 1.023 (inclusive). The fourth range of specific gravity values include values that are between 1.024 and 1.026 (inclusive). The fifth range of specific gravity values include values that are between 1.027 and 1.028 (inclusive). The sixth range of specific gravity values include values that are between 1.029 and 1.031 (inclusive). The seventh range of specific gravity values include values that are greater than 1.031 (inclusive). The second color 14b is darker than the first color 14a. The third color 14c is darker than the second color 14b. The fourth color 14d is darker than the third color 14c. The fifth color 14e is darker than the fourth color 14d. The sixth color 14f is darker than the fifth color 14e. The seventh color 14g is darker than the sixth color 14f.

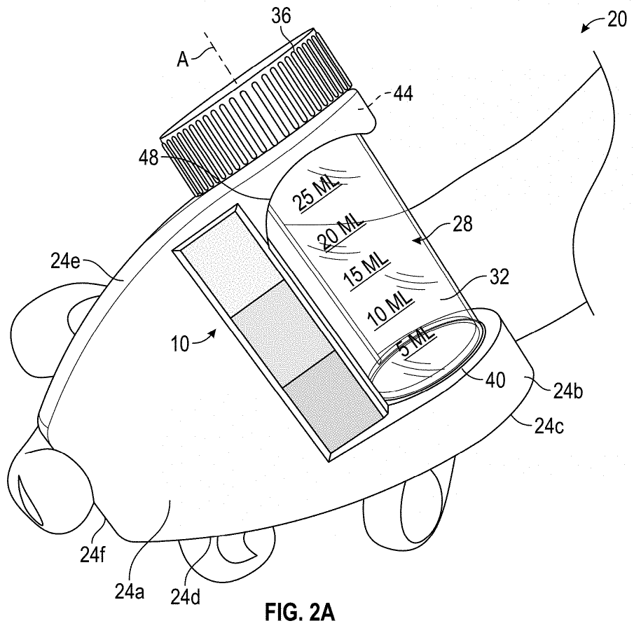

As shown in FIGS. 2A-2C, at least a portion of the color chart 10 of FIG. 1 may be used with a urine color and concentration assessment system 20. The urine color and concentration assessment system 20 may be used to assess undiluted urine by comparing the color of the undiluted urine to the colors of the color chart 10. In the illustrated embodiments, only three colors (e.g., the first, second, and third colors 14a-14c) are used with the color and concentration assessment system 20. This is because colors darker than the third color likely indicate a urine concentration representing dehydration. In other embodiments, more or fewer of the colors 14a-14g of the color chart 10 may be included.

As shown, the urine color and concentration assessment system 20 includes a housing 24 including a urine collection container 28. The housing 24 includes a first portion 24a that supports the color chart 10 and a second portion 24b that includes the urine collection container 28. The second portion 24b is positioned adjacent the color chart 10 such that at least a portion of a urine collection container 28 is positioned adjacent to the color chart 10. The housing 24 also includes a surface 24c that supports the housing 24 on a support surface (e.g., table top). In the illustrated embodiments, the housing 24 is formed as a single unitary piece. That is, the first portion 24a and the second portion 24b are integrally formed with one another. In other embodiments, the housing may be formed in other suitable ways.

In some embodiments, the urine collection container 28 may be integrally formed with the housing 24. In such case, the second portion 24b may be transparent or include a transparent portion such that a color of a urine sample retained in the urine collection chamber 28 is viewable.

In some embodiments, the urine collection container 28 may be separately formed from the housing 24. In some embodiments, like those shown herein, the urine collection container 28 may include a body 32 that is selectively sealed with a lid or closure element 36. In some embodiments, the urine collection container 28 may be removable from the second portion 24b of the housing 24. Regardless, the urine collection container 28 may have indicia indicating an amount of urine received therein.

In one embodiment, as shown in FIG. 2A, the second portion 24b of the housing 24 defines a recess 40 and an aperture 44 that are spaced apart from and coincident with one another. That is, in the illustrated embodiment, centers of each of the recess 40 and the aperture 44 are aligned along an axis A extending therebetween. In the illustrated embodiment, an opening 48 exists between the recess 40 and the aperture 44. In other embodiments, a transparent portion (e.g., a window made of transparent material) may cover the opening 48 on one or more sides. Regardless, the urine collection container 28 is positionable in the second portion 24b of the housing 24 and supported by the recess 40 and the aperture 44. In the illustrated embodiment, the urine collection container 28 may be supported relative to the housing 24 by an engagement between an edge of the lid 36 and a surface of the housing 24. That is, as shown in FIG. 2A, the lid 36 has a diameter that is greater than a diameter of the aperture 44, such that the lid 36 abuts a top surface 24d of the housing 24 when the container 28 is received in the housing 24. In other embodiments, the urine collection container 28 may be entirely positioned within and supported by the housing 24. That is, the lid 36 may have a diameter that is equal to or less than a diameter of the aperture 44, such that the lid 36 is positioned entirely or partially within the aperture 44 when the container 28 is received in the housing 24. Because the second portion 24b of the housing 24 (and specifically the opening 48) is positioned adjacent the color chart 10, the color of the urine contained within the urine collection container 28 can be compared with the colors 14a-14c of the color chart 10. Moreover, the housing 24 is configured to allow the urine sample to be exposed to a maximum amount of light. That is, the housing 24 (e.g., the second portion 24b of the housing) is configured to allow the user to view the urine sample from at least three sides. Although not shown in FIG. 2A, the urine color and concentration assessment system 10 may further include one or more light sources to illuminate one or both of the color chart and the urine sample. The one or more light sources may be positioned within the housing 24 or exterior to the housing 24. Alternatively, the housing 24 may be positioned adjacent a light source such that the light source may be positioned adjacent the container 28.

Further with respect to FIG. 2A, the second portion 24b includes the surface 24c that supports the housing 24. Moreover, in FIG. 2A, the surface 24c defines a portion of a bottom surface 24e of the housing 24. As shown, a portion of the bottom surface 24e is tapered relative to the surface 24c and relative to the axis A, such that a portion of the bottom surface 24e is spaced apart from the support surface when the housing 24 is supported thereon. Similarly, a portion of the top surface 24d is also tapered relative to the axis A. The first tapered surface and the second tapered surface, in part, form the first portion 24a. Also, as shown, the first tapered surface 24e and the second tapered surface 24d are tapered toward one another, such that the first portion 24a narrows on a side opposite the second portion 24b. In the embodiment of FIG. 2A, the first portion 24 includes a surface 24f that couples the first tapered surface 24e and the second tapered surface 24d. The surface 24f is positioned opposite the second portion 24b. The shape of the housing 24 may help the grasping the housing 24 when analyzing a urine sample. As shown, in the illustrated embodiment, the housing 24 has a first height defined by the second portion 24b and a second, smaller height defined by the surface 24f of first portion 24a. The housing 24, therefore, has as a variable height between the first height and the second height. The opening 48 defines a third height that is smaller than the first height, but greater than the second height. In the illustrated embodiment, the color chart 10 has a fourth height that is substantially the same height as the third height opening 48. Each of the three colors of the color chart 10 take up substantially a third of the fourth height. The term “substantially” is used in this paragraph to mean plus or minus ten percent.

In one embodiment, as shown in FIGS. 2B-2C, the first portion 24a of the housing 24 defines a bore 60 that is at least partially defined by a window 64 (e.g., formed from a transparent material). The bore 60 is aligned along an axis B. Similarly, the second portion 24b of the housing 24 defines a bore 72 that is at least partially defined by a window 76 (e.g., formed from a transparent material). The bore 72 is aligned along an axis C, which is parallel to the axis B. In other embodiments, the window 76 may be omitted, and an opening used instead. Regardless, the color chart 10 is positionable in the bore 72 in the first portion 24a of the housing 24 and is therefore visible via the window 64. Similarly, the urine collection chamber 28 is positionable in the bore 72 of the second portion 24b of the housing 24. In some embodiments, the bore 60 in the first portion of the housing 24 may be omitted and the color chart 10 integrally formed with or otherwise coupled to the housing 24. In the illustrated embodiment, the urine collection container 28 is entirely positioned within and supported by the housing 24. That is, a diameter of the lid 36 is equal to or less than a diameter of the bore 72, such that the lid 36 is positioned entirely or partially within the bore 72 when the container 28 is received in the housing 24. In the embodiment of FIG. 2B, the bore 72 includes recesses 72a in a circumference thereof, which facilitate the insertion of the user's fingers for inserting and removing the container 28 from the housing 24. In other embodiments, the urine collection container 28 may be supported relative to the housing 24, as discussed above with respect to FIG. 2A, if the lid 36 has a diameter that is greater than a diameter of the bore 42. Because the second portion 24b of the housing 24 (and specifically the window 76 thereof) is positioned adjacent the color chart 10, the color of the urine contained within the urine collection chamber 28 can be compared with the colors 14a-14c of the color chart 10.

Further with respect to FIG. 2B, the surface 24c defines the entire bottom surface 24e of the housing 24, and the bottom surface is parallel relative to the top surface 24. Additionally, in the embodiment of FIG. 2B, a front surface 24g of the housing 24 includes a recess 24h. A surface 24h′ of the recess includes the window 64. As shown, in the illustrated embodiment, the housing 24 has a single height between the top and bottom surface 24c, 24d. Each of the windows 64, 76 define a second height that is smaller than the first height. That is, the heights of the windows 64, 76 are substantially the same in the illustrated embodiment. Accordingly, a height of the color chart is substantially the same as the height of the window 76 through which the void of urine is visible. Like the embodiment of FIG. 2A, each of the three colors of the color chart 10 take up about a third of the second height.

Moreover, as shown in FIG. 2C, the urine color and concentration assessment system 20 may further include one or more light sources 80 to illuminate one or both of the color chart 10 and the urine sample. In the illustrated embodiment, a first light source 80 is positioned within the housing 24 adjacent to the color chart 10 in the bore 60 in the first portion 24a of the housing 24, and it is configured to illuminate the color chart 10 from behind the color chart 10 (e.g., by shining through an opening (not shown) in the bore 60). Similarly, in the illustrated embodiment, a second light source 80 is positioned within the housing 24 adjacent to the bore 72 in the second portion 24b of the housing 24, and it is configured to illuminate the urine sample from behind the urine sample (e.g., by shining through an opening (not shown) in the bore 72). Accordingly, in the illustrated embodiment, the light sources 80 are each positioned between the bores 60, 72 and a rear surface 24i of the housing 24. In other embodiments, the light source 80 may be positioned elsewhere relative to the color chart 10 and the urine sample to appropriately illuminate the color chart and the urine sample. For example, in another embodiment, the light sources 80 may be positioned between the bores 60, 72 and the bottom surface 24e of the housing 24, such that the light sources 80 illuminate the color chart 10 and the urine sample from the below the same. In yet another example embodiment, one of the light sources 80 may be positioned between the bore 60 and a first side surface 24j of the housing and one of the light sources 80 may be positioned between the bore 72 and a second side surface 24k of the housing 24, such that the light sources 80 respectively illuminate the color chart 10 and the urine sample from opposite sides of the same. Additionally or alternatively, in any of the embodiments discussed above, a light source could be positioned between the bores 60, 72.

The one or more light sources may be any suitable light source including, but not limited to, a halogen light source, a fluorescent light source, a light emitting diode (LED) panel, a light pipe, and/or a LED flashlight.

### Materials and Methods for Evaluating the Color Chart of FIG. 1

### Participants

An athletic population of 189 university National Collegiate Athletic Association (NCAA) Division I athletes in the USA, student club athletes, coaches, and tactical athletes (recruits of the Army Reserve Officer Training Corps, ROTC) (52% male, 22.3 1.6 years) participated. They reported being in good self-reported health and not using medications that could influence hydration status. Dietary supplement intake was checked, but participants using them were not excluded. Data were collected as part of a broader project that included the validation of a urine color scale to assess urine color directly from the toilet bowl. A Power of Sign Test revealed a 98% power to detect the distribution of samples below (58%) and above (42%) a selected USG threshold of 1.020 defining low vs. high urine concentration, respectively (with p=0.50 at α=0.05) based on this sample (n=189). The Institutional Review Board of Arizona State University approved the study (STUDY00010071).

### Urine Color Charts

To be able to better understand the accuracy of Uc self-assessments in an athletic population, and to generalize results, a mix of color charts derived from different methods, (e.g., charts based on ranking urine colors vs. charts based on urine concentration-based color categories) were evaluated. As many Uc charts are available, this approach helped to better understand the validity of urine color charts that were constructed based on different mechanisms.

### Development of the Urine Color Chart of FIG. 1

The 7-color urine color chart of FIG. 1 was based on a selection of 96 previously collected urine samples. The urine colors were based on the following USG ranges with (n) indicating the number of urine samples in each category: <1.017 (n=20), 1.017-1.021 (n=15), 1.022-1.023 (n=13), 1.024-1.026 (n=24), 1.027-1.028 (n=9), 1.029-1.031 (n=11), and >1.031 (n=4). This resulted in a range of 7 colors, with a very low concentration (score 1) to a very high concentration (score 7), consistent with the conventional categories for hydration status. A collective sample reflecting a urine concentration category was produced by combining an equal amount of each sample within the urine concentration category. This was done for each concentration category. Each newly produced color sample was aliquoted in a 30 mL tube without caps (30 mL free-standing Evergreen centrifuge tube, Caplugs, Buffalo, N.Y., USA), and placed in our specially designed scoring box. Tubes were lighted underneath by white LED flashlight (Ozark Trail, Ozark, Betonfille, Ark., USA) with 1848 lux measured using a foot-candle lux meter (Extech 407026, Extech Instruments, Waltham, Mass., USA) at the Tungsten/Daylight setting. Photos were taken of each collective sample with a digital single-lens reflex (SLR) camera, Canon EOS Rebel T4i with a Canon Zoom Lens EF-S 18-55 mm f/3.5-5.6 (Canon Inc., O⁻ ta, Tokyo, Japan). The camera was positioned 35.5 cm (14 inches) from the sample and placed behind a round 3.05 cm (1.2 inch) opening showing the sample against a white backdrop. White balance was set by taking a photo of a freestanding tube with 30 mL demineralized water. After digital photos were taken, the white color reference was confirmed using Adobe Photoshop (Adobe Inc., San Jose, Calif., USA) by identifying the whitest white and the blackest black. Then on-screen urine colors (Red-Green-Blue/RGB) were translated to a print color system. This resulted in the following Pantone Matching System (X-rite, Grand Rapids, Mich., USA), also called PMS colors (with hexadecimal (HEX) codes 0 short RGB color codes in parentheses), printed from left to right in rectangles size 5.1×7.6 cm (2×3 inch): Color 1: 7499 C (F1E6B2); color 2: 600 C (F1EB9C); color 3: 602 C (F0E87B); color 4: 603 C (EDE04B): color 5: 605 C (E1CD00); color 6: 7758 C (D4C304) and color 7: 124 C (EAAA00). Prints were made on a white paper measuring 43.2×27.9 cm (17×11 inch) with a gloss finish.

### Remaking a Conventional 8-Color Chart

The colors reported on the 8-color chart 100 described in the Dictionary of Color were used, as reported previously: color 1 17/B1; color 2 9/H1; color 31751; color 4 17/L1; color 5 9/13; color 69/L3; color 712/K6 and color 8 23/L1. These colors were digitally transferred to HEX codes and matte paints were selected with this exact code. This resulted in the following HEX codes and paint colors in parenthesis: color 1 FFFDD8 (DE 5407 Pumpkin seed (I), Interior velvet L Base); color 2 FFFBA8 (DE 5402 Lemon slice (I), Interior velvet L Base); color 3 FCE974 (DE 5417 Dandelion (I) (LH), Interior velvet M Base); color 4 FFBA00 (Yellow Finch 19D-5, Premium interior satin enamel); color 5 FFCE79 (DE 5290 Apricot glow (I), Interior velvet M Base); color 6 EAC853 (DE 5424 Yellow brick road (I) (LH), Interior velvet U Base); color 7 E1C161 (DE 5432 Candelabra (I), Interior velvet U Base); and color 8 898253 (DE 5496 Aged eucalyptus, Interior velvet U Base). All paints were Dunn-Edwards paints (Dunn-Edwards Corporation, Los Angeles, Calif., USA), except for color 4 being a Clark+Kensington paint (Ace Hardware Corporation, Oak Brook, Ill., USA). Each color was then painted onto wooden 2.5×15.2 cm (1×6 inch) rectangles and glued onto a 48.3×30.5 cm (19×12 inch) matte whiteboard with an equal 2.5 cm (1-inch) distance apart.

### Urine Color Scoring Box

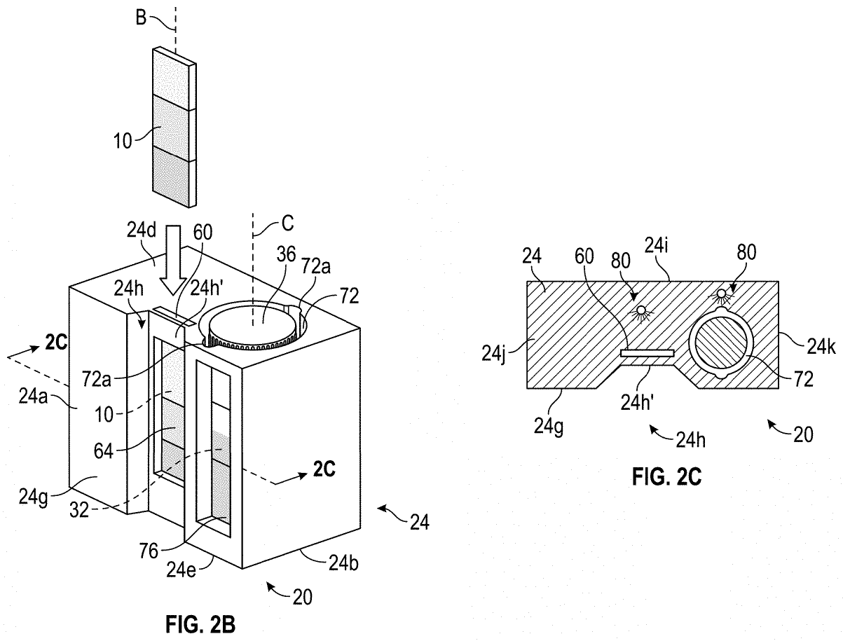

A box was constructed to standardize the participants' scoring (FIGS. 3A-3D). The box was black inside and was positioned on a white table, with the urine sample tube placed against a white backdrop 35.5 cm (14 inches) from a peephole 2.54×2.54 cm (1×1 inch) cut in the front of the box. To control lighting, a 28-watt color adjustable lamp providing an intensity of about 1650 lux at full power and light color set to white (NL480, Neewer, Shenzen, China) was placed on the left side of the box at the height of the sample, the scoring was done in a well-lit room directly under a 3-light fluorescent parabolic troffer (420 lux) built into the ceiling.

### Procedures

### Urine Color Scoring

Black cups were provided to each participant and voiding time was registered to the nearest second and noted on the container lid. If their urine sample was not collected at a predetermined collection site, participants were instructed to perform Uc testing within four hours of collection. Participants handed in their urine sample and recorded their study ID, time collected (first morning or later), voiding duration (sec), sex, age (years), and type of athlete affiliation. Body height (cm) (Seca 213 portable stadiometer, Hamburg, Germany) and body weight (kg) (Seca 803 digital scale, Hamburg, Germany) were measured. A research team member prepared three separate 1.5 mL tubes for measuring urine osmolality, as well as a 30 mL urine sample (30 mL free-standing Evergreen centrifuge tube, Caplugs, Buffalo, N.Y., USA) for Uc scoring. Each sample was covered using clear Parafilm (Laboratory Film, Bemis Company Inc., Neenah, Wis., USA) to seal and prevent color distortion. With respect to FIG. 3E, participants were then instructed to look into the box and score their sample using the two color charts (FIGS. 3F and 3G). The color charts of FIGS. 3F and 3G were alternately used for scoring by participants based on the chart that their predecessor scored first.

### Urinalysis

Urine collection cups were measured before and after collection of the sample on a precision scale with 0.1 g accuracy (PT 1400, Sartorius A G, Göttingen, Germany), and so true urine weight could be obtained with the assumption that grams of urine could be translated to milliliters. After Uc was scored, both the 1.5 mL as the 30 mL urine samples were stored at 5° C. until further analysis for urine concentration. Urine can be stored at fridge temperature (5° C.) for 5-7 days without a change in concentration.

Urine specific gravity was measured in fresh urine samples (stored no longer than five days in the refrigerator) using a USG refractometer pen (Pen-Urine S. G., Atago, Tokyo, Japan) at a sample temperature of 20° C. Each measurement was performed twice; in case a variance larger than 0.0005 was detected between the two measurements, a third measurement was added and the median was calculated. Duplicate measurements were performed to calculate mean urine osmolality (with sample CV 0.17±0.18) in fresh urine samples (stored no longer than seven days at a temperature of at 5±1° C.) using freezing point depression (A2O Osmometer, Advanced Instruments, Norwood, Mass., USA).

### Statistical Analyses

Personal characteristics (height: m, weight: kg), urine volume (mL), urine voiding time (sec), USG, osmolality (mmol kg⁻¹), and Uc were reported as medians and interquartile range, or when appropriate as mean standard deviation (sd). Associations based on Spearman correlation coefficients r (including 95% CI using Fisher's Z transformation) are reported for urine color, -voiding time, -volume, USG, and osmolality.

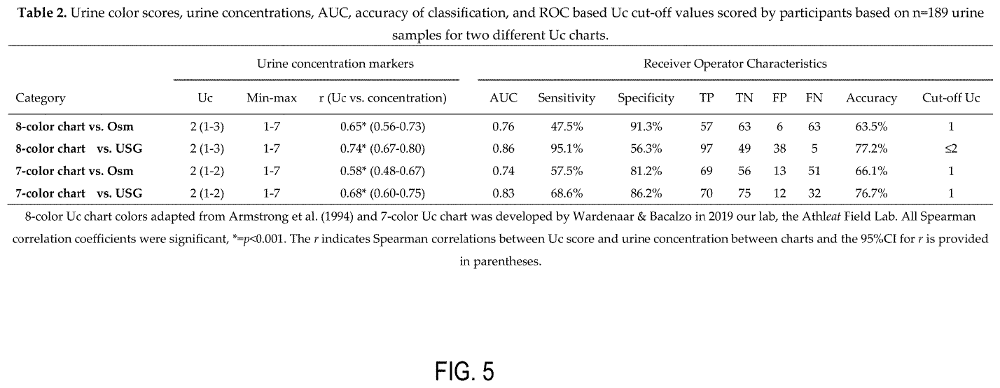

To address aim (a), to determine differences in scoring Uc for two different charts 10, 100, mean differences were analyzed via Mann-Whitney U tests. Spearman's correlation coefficient tests correlated Uc scores against urine concentration. A Bland-Altman plot (FIG. 6) was produced comparing Uc scores from 1-7 (as score 8 was not reported using the 8-color Uc chart) to evaluate charts against each other. To assess whether Bland-Altman results were biased between charts for scoring Uc lighter, similar or darker, a Spearman correlation coefficient was calculated using the Bland-Altman results from the y-axis (difference between reported Uc outcomes) vs. x-axis (means of both outcomes). The level of agreement is expressed as M(_{difference}) 1.96 SD(_{difference}). Statistical significance was accepted at p≤0.05.

To address aim (b), to determine the color charts' diagnostic ability to assess under-hydration based on the correct classified urine samples for urine concentration, receiver operating characteristics (ROC) curves were calculated. This resulted in the calculation of the area under the curve (AUC), sensitivity, specificity, optimal Uc threshold score and accuracy of correct Uc scores (%) in classifying urine concentration, as well as true positive (TP), true negative (TN), false positive (FP), and false negative (FN) scores. When interpreting the area under the curve an AUC≥0.90 is considered excellent, 0.80-0.89 is considered good, while an AUC of 0.70-0.79 is to be considered fair. Sensitivity and specificity scores are preferred to be above 0.80. Sensitivity is defined as the number of true positive (TP) scores suggesting underhydration, divided by the sum of TP and false-negative (FN) scores. Specificity is defined as the number of true negatives (TN) divided by the sum of false positives (FP) and TN. To classify urine samples with a low vs. high concentration, the optimal Uc threshold score to predict underhydration was determined from the area under the curve (AUC) using the max approach of the sensitivity and specificity matching the selected urine concentration cut-off values for USG <1.020 and osmolality<800 mmol kg⁻¹. As dietary supplement use may influence Uc scoring, a stratified analysis was performed to assess the accuracy of Uc scoring splitting supplement users and non-users.

### Results

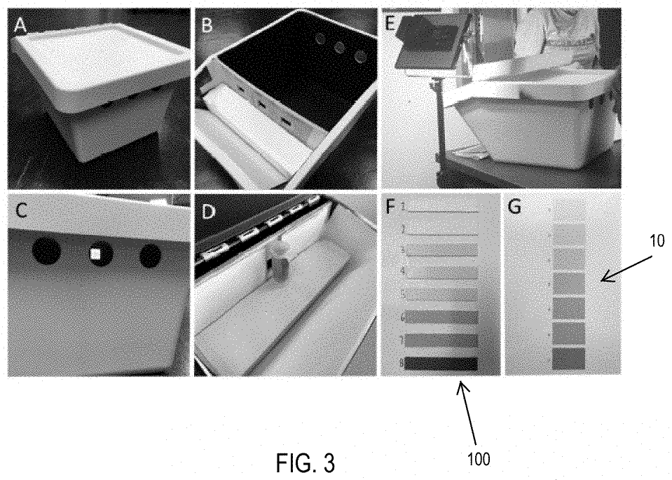

As shown in Table 1 of FIG. 4, of the 189 participants, most were White (n=105), most were student-athletes (n=132), and about half were female. Visiting Chinese coaches (n=24) were substantially older (37 years, 34 years to 39 years) than the rest of the population (20 years, 19 years to 22 years). The overall median urine volume was 248 mL (138 mL to 391 mL), 58.7% of the participants delivered a first morning sample, and the median voiding duration was 16 seconds (11 seconds to 25 seconds). The total sample had a median osmolality of 705 mmol kg⁻¹ (465 mmol kg⁻¹ to 930 mmol kg⁻¹) and urine specific gravity of 1.019 (1.013 to 1.024). Correlations between urine osmolality and urine specific gravity ranged between r=0.80-0.97, and all correlations reported in Table 1 of FIG. 4 were significant (p<0.001).

To answer aim (a), to determine differences in scoring Uc between both charts, the 8-color Uc chart scored Uc significantly darker than the 7-color Uc chart (2.2±1.2 vs. 2.0±1.2, respectively, p<0.001). There was a moderate correlation between Uc charts (r=0.76, 95% CI: 0.69-0.81). The Bland-Altman plot (FIG. 6) shows that 60.5% of the scores of the 8-color and 7-color Uc charts were similar. When comparing scores 1 color shade, similarity was 96.5%. There was a significant weak reporting bias (p=0.04) indicated by r=0.15 between both Uc charts, indicating that the 7-color Uc chart resulted in slightly lighter scoring than the 8-color Uc chart. With respect to FIG. 6, the size of the bubbles indicates the percentage of urine color scores, small black dots represent 1% values, and circled dots represent 2-3% values. The average reporting bias is indicated by the dotted line denoted +0.2±0.8, and the 95% limits of agreement are indicated by the dotted lines denoted +1.7 and −1.3.

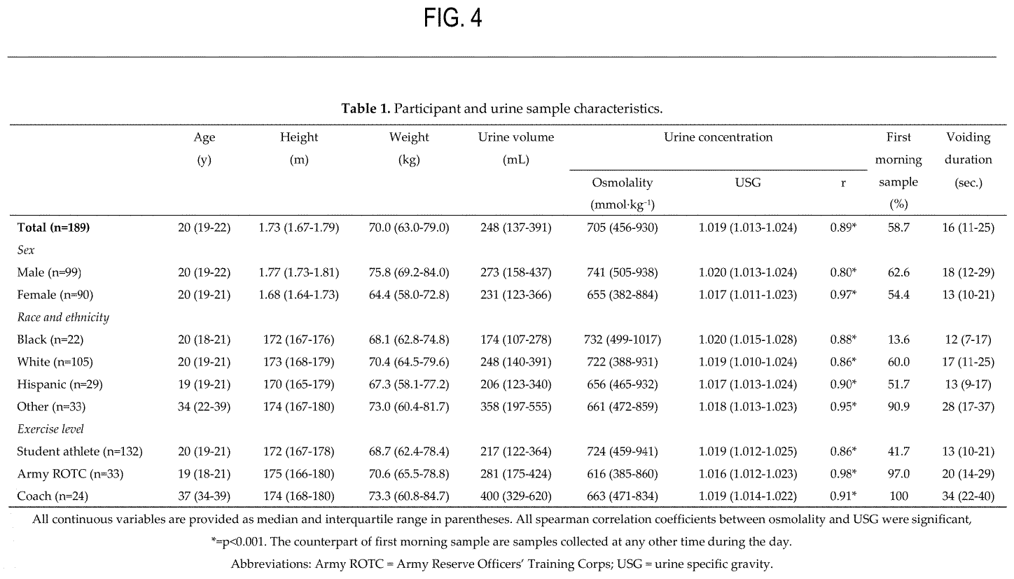

As to aim (b), determining the diagnostic ability of the two Uc charts vs. urine concentration measures, data in Table 2 of FIG. 5 suggest the diagnostic ability of the 8- and 7-color charts on a group level is fair in relation to osmolality (0.76 and 0.74, respectively), and good in relation to USG (0.86 and 0.83, respectively). Sensitivity was low and specificity was high when Uc scores were related to osmolality. USG values for sensitivity and specificity were less consistent. For the 8- and 7-color Uc charts, the accuracy of Uc against urine concentration was higher for USG (77.2% and 76.7%, respectively) compared to osmolality (63.5% and 66.1%, respectively). The optimal cut-off Uc, needed to accurately classify urine samples for a low vs. high concentration, was 1 when both Uc chart scores were compared with osmolality. When compared to USG, the 8-color Uc chart resulted in a cut-off Uc value 2 while the 7-color Uc chart had the best fit for a Uc of 1 to detect low vs. high urine concentration.

Finally, no clear differences for the AUC were seen when stratified analysis was performed for sex. Both men and women reported consistently with the AUC reported on group level. An additionally stratified analysis assessing the AUC and the correct classification of urine samples for participants using dietary supplements (n=28) vs. those not using them (n=161) showed no substantial difference between groups. The AUC for supplement users (0.85) was actually higher for both Uc charts vs. non-users (0.82 for the 8-color Uc chart, and 0.80 for the 7-color Uc chart). The accuracy of correct scored urine samples for dietary supplement user and non-users was slightly reversed for the 8-color Uc chart (71.4% vs. 74.5% accuracy, respectively), but not for the 7-color Uc chart (75.0% vs. 73.9% accuracy, respectively).

### Discussion

This study is the first to report the validity of a self-assessment of traditional multi-shade Uc assessment in an athletic population using different Uc charts to classify low vs. high urine concentration. This knowledge will help athletes determine whether to increase fluid consumption on a daily basis. Additionally, the results will inform health professionals about the strengths and limitations of this method. The diagnostic ability of the charts expressed by the AUC and the accuracy of correct classified urine samples was fair (8-color Uc chart) to good (7-color Uc chart). Depending on the type of chart used, athletes may report slightly darker Uc scores when scoring urine samples based on the 8-color vs. the 7-color Uc chart. Finally, the self-reported accuracy was almost 10% higher when Uc scores were compared to a USG cut-off value of 1.020 vs. a urine osmolality cut-off value of 800 mmol kg⁻¹.

The osmolality-based AUC reported in this study was 0.76 for 8-color Uc chart and 0.74 for 7-color Uc chart, similar to self-reported values (0.67-0.78) in children. The USG-based AUC was 0.74 for 8-color Uc chart and 0.83 for 7-color Uc chart, which is equal to or higher than an earlier USG value of 0.73 self-reported in females. Finally, a similar AUC for Uc scoring was found in a recently validated lavatory method assessing urine color from the toilet bowl using diluted color shades, with osmolality (0.73) and USG (0.76). This suggests that an AUC between 0.74-0.86 is to be expected from untrained individuals scoring the color of urine sample regardless of the chart method used. Overall, laypersons may report slightly lower AUC values than trained investigators reporting a lab technician-based AUC of 0.96. This suggests that when properly trained, athletes can potentially improve the accuracy of their scores.

Significantly more “Uc 2” scores were recorded for the 7-color Uc chart vs. more “Uc 3” scores for the 8-color chart, which could be due to some small color differences in construction between charts. The relatively small tubes (30 mL) exposed to bright LED light, equal to about 1650 lux, are likely to report 1-2 shades lower cut-off values than previously reported based on samples scored in a “well lit” room. At this light intensity, both charts reported relatively similar results, but it could be speculation if charts are comparable when used under different light conditions, as FIGS. 3F and 3G show visual differences between the darker color panels on the charts. Overall, when focusing on the collected results the study shows that, despite minor differences in reporting between the two Uc charts, results for both charts are comparable.

Earlier studies reported optimal cut-off values for 8-color Uc charts between 3 and 5, while the current study reported values of 1 and 2. Based on the current study results, coaches and athletic training staff should inform their athletes that multi-shade Uc charts should be viewed as a sliding scale rather than a single cut-off value depending on the light conditions in which the urine sample it scored.

There was a difference in the accuracy of classifying samples for hypohydration between osmolality and USG. This difference could be the result of a slight mismatch between the selected cut-off values. The selected cut-off value for osmolality (800 mmol kg⁻¹) marked the suggested onset of dehydration, comparable to a USG value of 1.020 based on 24-hour urine collections. Whereas others used a slightly lower cut-off of 700 mmol kg⁻¹ to define hypohydration. The differences in Uc scoring accuracy is therefore likely the result of the two cut-off values diagnosing slightly different hydration states, i.e., USG assessing a lower non-clinical level of hypohydration and the somewhat higher urine osmolality concentration assessing a more progressed form of non-clinical hypohydration.

Although the literature is not consistent about the impact of dietary supplements on urine color scoring, there is some evidence that riboflavin (B₂) influences Uc scoring in a negative way. However, no substantial differences were found between correctly classifying low vs. high urine concentrations, based on the calculated AUC for samples provided by supplement users and non-users.

This study was not without its limitations. The number of color shade panels between charts were different, but none of the samples was scored color 8 suggesting that this was not a large limitation while comparing both charts during this data collection. There was no standardization of fluid intake and spot urine samples were collected at various times during the day. Sport morning samples are known to have a somewhat higher concentration than 24-hour urine collections. On the other hand, the majority of the samples were collected in the afternoon hours, with urine concentrations closely related to 24-hour urine collections. Additionally, Uc and urine concentration can be influenced by acute rehydration strategies after practice, which could lead to a mismatch between Uc and urine concentration due to acute dilution of the urine. No time was determined between the last practice in relation to urine collection. It is also likely that the study was impacted by recruitment bias, triggering mainly those interested in assessing their hydration status. Despite a diverse population, the distribution was somewhat skewed with largest part of the population existing of White student-athletes, with fewer Black, Hispanic, and other participants. Finally, this validation was performed in a controlled setting, therefore generalizations towards other use of Uc charts in a different setting need to be made with caution.

Considerations for practitioners include that a high urine concentration is associated with underhydration, a phenomenon in which a low water intake is associated with high vasopressin levels and urine concentration, without bodyweight change or a sensation of thirst. The assessment of Uc can help to identify athletes with a low vs. high fluid intake. This is a simple assessment that can be used in multiple settings, before regular practice, at home, or while travelling. Additionally, hydration assessment can be of use when assessing body composition such as bioimpedance measurements, or when performing exercise testing allowing for a better standardization of measurements.

Despite the small significant difference in reporting Uc, no real practical difference exists between the two validated Uc scales when looking at the total number of correctly classified urine samples (up to 77%). When looking deeper into this misclassification, the 7-color Uc chart showed a much lower number of false-positive classifications (6%) than the 8-color chart (21%). This is an important difference, because false-positive classifications as a result of a light urine color score, while the concentration was actually above the selected urine concentration cut-off value, would likely not prompt an athlete to increase fluid intake. At the same time, the numbers for false-negative classifications were reported in reverse, resulting in a larger number of athletes being prompted to drink by the 7-color Uc chart. This highlights the importance of educating athletes on the proper timing of hydration and rehydration strategies, including advice about drinking volume to ensure safe drinking practices. Preferably, the assessment of Uc should be combined with other methods to allow for a better detection of a suboptimal hydration status. A good example is that a combined assessment of urine color and urine void frequency during a 24-hour period results in a 97% diagnostic ability for underhydration.

### Conclusions

The results suggest that the use of two multi-shade Uc charts (one 8-color and one 7-color), regardless of the difference in method for constructing their color shade panel, is similar to Uc self-assessments by athletes. A greater classification accuracy for low vs. high urine concentration occurs, up to 77% correct classified samples, when Uc scoring is compared to urine specific gravity rather than urine osmolality.

### Validity of Scoring Urine Color at Different Light Intensities as a Diagnostic Tool to Assess Low Versus High Urine Concentration

Urine color (Uc) is used to assess urine concentration when lab techniques are not feasible. This study aimed to compare the accuracy of Uc scoring using different light conditions with a 7-color Uc chart. As discussed in greater detail below, 178 previously frozen urine samples were scored under eight conditions (four light sources, and two scoring techniques, the traditional over a chart vs. in a scoring box). A receiver operating characteristics (ROC) analysis was performed to compare the diagnostic capacity against a 1.020 urine specific gravity (USG) cut-off, resulting in Uc scoring accuracy and optimal Uc cut-off value. To assess the results' generalizability, the outcome of a subsample (n=78) was compared to fresh samples. Uc was significantly different between light conditions (P<0.01), with the highest accuracy (80.3%) of correctly classifications of low or high urine concentrations occurring at the brightest light condition. Lower light intensity scored 1.5-2 shades darker on a 7-color Uc scale than bright conditions (P<0.001), with no practical difference between scoring techniques. Frozen was 0.5-1 shade darker than freshly measured Uc (P<0.004), but they were moderately correlated (r≥0.64). A Bland-Altman plot showed that reporting bias mainly affects darker Uc without impacting the diagnostic ability of the method. Uc scoring and accuracy is affected by lighting condition but not by scoring technique, with higher accuracy and a one-shade lower Uc cut-off value at the brightest light. This suggests that the Uc cut-off value needs to be adjusted to light conditions to optimize scoring accuracy.

There is growing evidence of the long-term health benefits from fluid intake that results in a urine concentration of ≤500 mOsm/kg and a urine specific gravity (USG) value ≤1.012. As spot morning urine samples generally have a higher concentration than a full 24-hour urine sample, the cut-off value to assess a well-hydrated status potentially lies somewhat higher. Therefore, concentrations of 700 mOsm/kg or 1.020 USG have often been reported. The most accurate way to assess urine concentration involves lab-based techniques. Still, urine color (Uc) has been suggested as an appropriate proxy measurement in an applied setting to assess the hydration status of patients that are hospitalized, in nursing homes, or community-dwelling and others that want to assess their hydration status at home. There are several advantages to measuring Uc: the method is inexpensive, non-invasive, does not require technical expertise, and gives immediate results.

Urine color (Uc) charts have been valuable in hydration assessment and education in many settings, including clinical, athletic, and household. However, the main disadvantage of using the current common Uc charts is their lack of sensitivity (˜80% sensitivity accuracy). Sensitivity is calculated as the number of true positive (TP) scores suggesting a high urine concentration, divided by the sum of TP and false-negative (FN) scores. Despite the linear relationship between urine concentration and Uc, the sensitivity of Uc scoring (˜0.80) is suboptimal. Certain variables, such as vitamins or other bioactive substances, as well as protein, can influence its color 17 or concentration. On the other hand, the accuracy of correctly predicting urine concentration based on Uc can be improved substantially when using spectrophotometry, resulting in an analysis with 97.4% sensitivity. Showing that assessing light-based urine concentration with high accuracy is possible, but it could be asked how visual analog Uc scoring can be optimized by altering light conditions to improve the sensitivity of self-reporting assessment.

The correct classification of urine samples as low or high urine concentration may depend on environmental conditions that influence how the color or the urine sample is perceived. Earlier studies on Uc have reported a wide range of average Ucs for different populations. At the same time, multiple “optimal” Uc cut-off values, varying between ≤3 and ≤5, have been suggested for a traditional 8-color Uc chart. Apart from the diameter (volume) and materials used in the urine container, the type of light and the light intensity will influence perceived Uc. Beer-Lambert' s law states that light absorbance is equal to the concentration of the solution the light is passing through, the solution's volume, and the absorption coefficient. Despite studies reporting that urine samples were scored in a well-lit room, not much information is available about the actual light conditions used and how the urine sample was positioned against the light to be scored for its color.

As previous studies have not clearly defined their light conditions on which the suggested Uc cut-off values have been based, health professionals, such as clinicians, nurses, dietitians, and athletic trainers, would benefit from more information on what Uc cut-off value would be appropriate given different available light types. Such insight will help further standardize the Uc scoring process, potentially resulting in better self-classification of urine samples with low or high urine concentration. This information will allow them to instruct their clients or patients on interpreting the Uc score results to more accurately determine hydration status. Therefore, the objective of this study was to compare the accuracy of Uc scoring by investigators based on previously frozen urine samples at different light conditions with a range of light types and intensities. The aims were as follows: (a) to report Uc scores for four light condition using a 7-color Uc chart and two different scoring techniques resulting in eight different Uc scoring combinations, (b) to investigate the diagnostic ability and the optimal Uc cut-off value for each of the eight scoring combinations to assess Uc, and (c) perform a sensitivity analysis to compare the results of a subsample (n=78) before and after they were frozen to determine if the results from aim (a) and (b) can be generalized to freshly collected and measured Uc.

### Methods

### Design

To evaluate Uc scoring differences between four different light conditions, we used the 7-color Uc chart and two scoring techniques to explore potential differences between how samples are scored under different light conditions. The first method entailed comparing the tube directly with the chart. The second aimed to further standardize the Uc scoring process by fixing the distance from sample to the observer and the light angle, using a Uc scoring box for sample scoring. Three research technicians individually scored each sample, then the calculated median Uc scores were compared with the measured specific gravity (USG) of initially fresh urine to identify the accuracy for correctly classifying urine samples with a low vs. high urine concentration for each condition. Urine samples were classified as having a low urine concentration with a USG cut-off value <1.020 and a high urine concentration above this value. As frozen samples tend to display a slightly darker color, a multilevel comparison was performed between a subset of the samples measured before they were frozen, as part of the original data collection, and their color scores after they had been frozen. This analysis allowed for better insight into comparing the frozen samples with samples measured in a fresh condition.

### Sample Number

The 178 urine samples were initially collected between 2018-2019 during three studies previously approved by the Institutional Review Board at Arizona State University: STUDY00010071, STUDY00008336, and STUDY00007260. Urine samples were anonymized before starting the project. Apart from the original measured urine specific gravity value for each sample, no other data were carried over to this study. Samples were collected as a spot urine sample and stored in 30 mL tubes at −20° C. At the start of this study, all samples received new identification numbers (U1-U178).

### Procedures

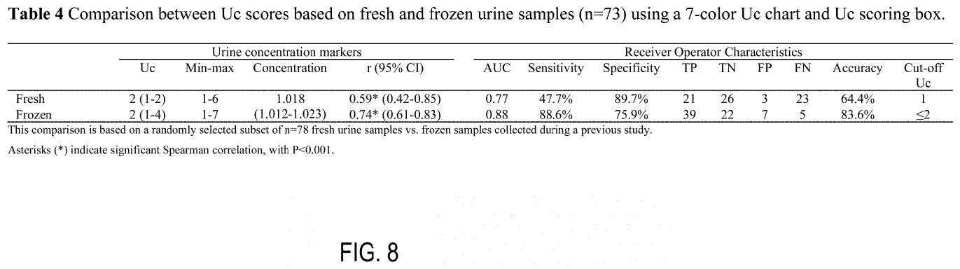

During each scoring session, ˜30 samples were selected. The frozen samples were thawed until they reached room temperature (20° C.) for scoring. Green lids were removed from the centrifuge tubes and replaced with clear film. Two folding tables were set up alongside each other to set up four scoring stations, each with one light condition (i.e., office light, restroom light, or one of two LED lights). The four stations, one per light condition, each included two scoring techniques to score the samples. The research technicians were accompanied by one assistant to help coordinate the samples' transfer from one person to the next. Per scoring round, each of the technicians received an equal number of samples splitting the total into three smaller batches. After scoring a batch of samples, samples rotated while technicians remained at their station with the scoring method and light type. FIG. 9 illustrates all scoring combinations. Per scoring day technicians scored all selected samples at a light station, before moving over to the next light condition. The scoring and rotating urine sample batches started again until all samples were scored. All technicians scored all samples under all eight conditions.

### 7-Color Uc Chart

The new 7-color color chart 10 of FIG. 1 was based on a selection of 96 previously collected urine samples. The Uc categories were based on the following USG ranges with (n) indicating the number of urine samples in each category: 131<1.017 (n=20), 1.017-1.021 (n=15), 1.022-1.023 (n=13), 1.024-1.026 (n=24), 1.027-1.028 (n=9), 132 1.029-1.031 (n=11), and >1.031 (n=4). This resulted in a range of 7 colors, with a very low concentration (score 1) to a very high concentration (score 7), consistent with the categories for hydration status suggested by Armstrong et al. (2010). Photos were then taken with a digital SLR camera, Canon EOS Rebel T4i with a Canon Zoom Lens EF-S 18-55 mm f/3.5−5.6 (Canon Inc., Tokyo, Japan), of each sample reflecting a urine concentration category that was produced by combining an equal amount of each sample within each of the urine concentration categories. Then each of the newly produced color samples was aliquoted in a 30 mL tube without caps (30 mL free-standing Evergreen centrifuge tube, Caplugs, N.Y., USA), while being placed in specially developed Uc scoring box. Tubes were lighted underneath by white LED light with 1848 lux. The camera was positioned 35.5 cm (14 inches) from the sample, and placed behind a round 3.05 cm (1.2 inch) opening showing the sample against a white backdrop. To set a white balance reference, a photo was taken of a freestanding tube with 30 mL demineralized water. After digital photos were taken, the white color reference was confirmed using Adobe Photoshop (Adobe Inc., San Jose, Calif., USA) by identifying the whitest white and the blackest black. The next step was to translate the on-screen urine colors (Red-Green-Blue/RGB) to a print color system; the primary choices are CMYK (Cyan-Magenta-Yellow-Key/Black) and Pantone Matching System (PMS). The primary difference between the two systems is when the ink is mixed and applied to the paper; more details regarding the PMS system and its colors can be found at www.pantone.com. Adobe Photoshop contains built-in color swatches to select from, which allows for a very quick and efficient translation from RGB to PMS. This resulted in the following PMS colors (with HEX codes 0 short RGB color codes in parentheses), printed from left to right in rectangles size 5.1×7.6 cm (2×3 inch): color 1 7499 C (F1E6B2); color 2 600 C (F1EB9C); color 3 602 C (F0E87B); color 4 603 C (EDE04B): color 5 605 C (E1CD00); color 6 7758 C (D4C304) and color 7 124 C (EAAA00). Prints were made on a 43.2×27.9 cm (17×11 156 inch) white paper with a gloss finish.

### Scoring Techniques

The accuracy of two different techniques was evaluated. The ‘traditional’ sample over chart method, as previously described, and the use of a specially designed Uc scoring box aimed to explore if further standardization of scoring distance and light angle could lead to a higher accuracy compared to the traditional scoring method. Urine samples were prepared in a 30 mL transparent plastic centrifuge tube (free-standing Evergreen centrifuge tube, Caplugs, N.Y., USA). Each urine sample was covered using clear Parafilm (Laboratory Film, Bemis Company Inc, Wis., USA) to seal the sample and prevent color distortion, allowing the scoring of samples without the original green tube cap. Each urine sample was inverted three times before being scored. Sample over chart method: During this method, technicians slid each separate urine sample over the white part of the Uc chart, comparing the sample with each color on the 7-color Uc chart.

Then a decision was made about the color of the samples, and the score was noted. Uc box scoring method: The second method involved a color scoring box constructed in the lab. This box was constructed to standardize the distance from the observer to the sample, and the way the urine sample was positioned in relation to the light for each separate light condition. The box (41×55×28 cm) with lid covering a 35.5 cm (14 inches) distance to the urine sample (Sortera, IKEA, Delft, The Netherlands) was black inside. It was positioned on a white table, and the urine sample tube was placed at the part of the box that was not covered with a lid against a white backdrop in front of the center peephole 2.54×2.54 cm (1×1 inch) cut in the front of the box. For this study, the box was slightly modified for the light conditions that did not involve LED flashlight scoring, allowing for a full white underlayment under the urine samples covering the built-in LED flashlight. Technicians looked through the box while comparing the color they perceived with the colors on the 7-color Uc chart positioned directly aside the box. A decision was made about the color of the samples, and the score was noted.

### Light Conditions

Light conditions were selected based on practical relevance. The light intensity was different for each light condition, but was similar for scoring techniques within each light condition. Each light condition intensity was measured using a foot-candle lux meter (Extech 407026, Extech Instruments, Waltham, Mass., USA) at the Tungsten/Daylight setting. The LED light conditions represent a combination of light (as background lights were not turned off).

Halogen: The restroom halogen light was part of the testing facility. The lights were built into the ceiling 6 feet apart in a square cornering all ends of two tables that created the testing station. The light intensity for this condition was 224 lux, measured at the center of the testing station, with the surface of the tables 180 cm (6 feet) from the ceiling.

Fluorescent: The lab space had fluorescent office light. The two 3-light fluorescent parabolic troffers were built into the ceiling 6 feet apart, matching the long end of the testing stations ensuring that measurements were performed directly under the light source. The light intensity for this condition was 402 lux, measured at the center of the testing station, with the surface of the tables 6 feet from the ceiling.

LED panel: For the sample over chart method, the 28-Watt LED panel light (NL480, Neewer, 00 Shenzen, China), providing 1666 lux, was placed 12 cm (7.5 inches) on the left side of the sample. For urine color box scoring, the light was placed on the left side of the 30 mL urine sample and the scorer's perspective. In all cases, the light was set to full white. The LED panel analysis was done in the restroom facility with halogen ceiling lights, as previously described, switched on.

LED flashlight: The flashlight contained six LEDs (Ozark Trail, Ozark, Ark., USA), providing 1848 lux when covered with a single layer of white masking tape to create a filter. The light was projected directly from underneath the 30 mL centrifuge tube for both scoring methods. During the sample over the chart method, technicians wore blue lab gloves and held the urine sample directly on the flashlight. For the color box scoring, the flashlight was built into the box to light up the sample from underneath when placing it in the center of the box on top of the flashlight. The LED flashlight analysis was done in the office space with fluorescent ceiling lights, as previously described switched on.

### Statistical Analysis

The urine concentration from the freshly measured samples was reported as the median and interquartile range (IQR). To address aim (a), to evaluate the scored Uc using different lights and techniques, median, and interquartile range (IQR), as well as mean and standard deviation (SD) were reported for each scoring condition to allow for easy comparison of the results as in the literature Uc is reported as mean or median depending on the publication. Differences between and within light conditions, on a group level, were assessed using Friedman and Wilcoxon signed-rank tests.

To address aim (b), investigating the diagnostic ability under the eight conditions to distinguish between low and high urine concentration based on the correct classification of Uc receiver operating characteristics (ROC) curves were calculated. Uc scores were optimally fitted against USG values that were initially measured in fresh urine samples. The best Uc cut-off value to distinguish urine samples with a low vs. high urine concentration (<1.020 USG cut-off value) was determined from the Area Under the Curve (AUC) using the max approach for sensitivity and specificity. An AUC ≥0.90 is considered excellent, 0.80-0.89 good, and an AUC of 0.70-0.79 fair, with sensitivity and specificity preferably above 0.80. Sensitivity and specificity were based on the number of true positive (TP: scoring a high Uc, suggesting a high urine concentration), true negative (TN: scoring a low Uc, suggesting a low urine concentration), false positive (FP: scoring a high Uc, suggesting a high urine concentration while the actual concentration is below the selected cut-off) and false negative (FN: scoring a low Uc, suggesting a low urine concentration while the actual concentration is above the selected cut-off) scores against a 1.020 USG cut-off value. Sensitivity was defined as the number of true positive (TP) scores divided by the sum of TP and false negative (FN) scores 15, and specificity was defined as the number of true negatives (TN) divided by the sum of false positives (FP) and TN.

To address aim (c), investigating the generalizability of the study outcomes based on frozen samples before analysis, we compared data from a subsample (n=78) measured prior to their freezing and measured after freezing. The original USG value of freshly measured samples was reported and correlated against frozen using Spearman's correlation, including 95% CIs using Fisher Z-transformation. The mean difference between frozen and fresh samples was analyzed through the Wilcoxon signed-rank test and Spearman's correlation. Further, a Bland-Altman plot was produced to assess the agreement between scores for individual samples, comparing Uc scores from 1-7 to evaluate the outcomes from freshly scored urine samples against frozen samples. To assess whether Bland-Altman results were biased for scoring Uc lighter, similar, or darker between fresh and frozen urine samples, an additional Spearman correlation coefficient was calculated. This analysis was done correlating the Bland-Altman results from the y-axis (the difference between reported Uc outcomes) against the results of the x-axis (means of both outcomes), indicating reporting bias when the correlation was significant. The level of correlation provided more information about the direction of this bias. Finally, the agreement level was calculated, defined as M(difference)±1.96 SD(difference). Statistical significance was set for all analyses at P≤0.05.

### Results

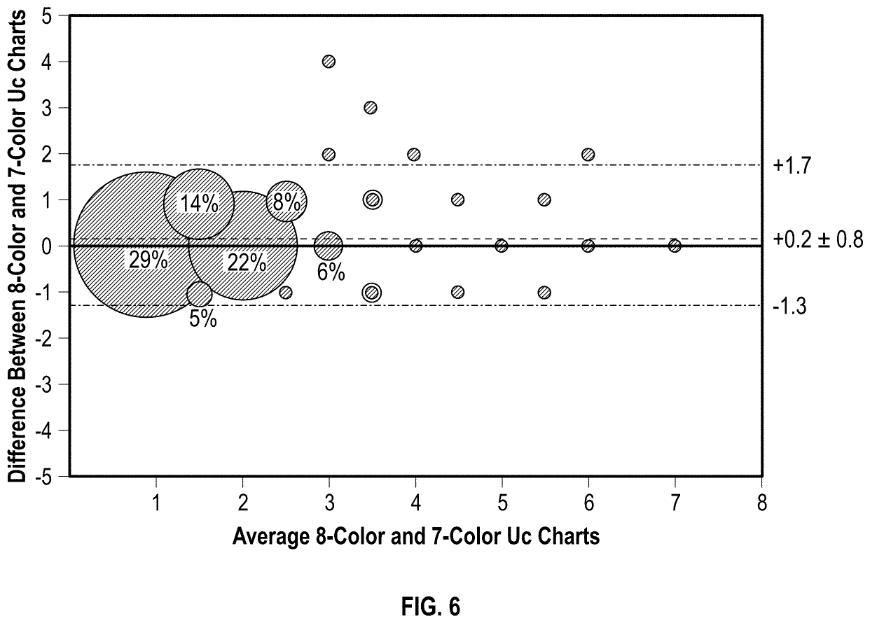

The original median USG value measured in the n=178 fresh samples was 1.018 (IQR 1.012-1.027). The outcome for aim (a), scoring Uc using different lights and techniques, is reported in the Table 3 of FIG. 7, which shows median and IQR scores. The average of the combined median values per scoring technique resulted in a significant difference for Uc between all light types (P<0.001). The conditions with lower light intensity, such as halogen and fluorescent, scored 1.0-1.5 shades darker than the LED light conditions. The average Uc for each scoring techniques for fluorescent light was 3.5 (2.0-6.0), for halogen light 3.0 (2.0-6.0), the LED panel 2.0 (1.0-5.5), and the LED flashlight 2.0 (1-4). Despite the higher light intensity, fluorescent light reported the darkest Uc, before halogen and the brighter LED conditions. Scoring techniques, i.e., sample over chart vs. in scoring box, significantly influenced scoring under each light type (P≤0.01), with the exception of halogen light (P=0.91).

The AUC, calculated to investigate the diagnostic ability of the eight measured conditions as formulated for aim (b), slightly increased with brighter light conditions. The lowest AUC for the over chart and Uc scoring box, for fluorescent light was 0.82 and 0.84, respectively, followed by halogen light (0.84 and 0.86), LED light panel (0.87 and 0.84), and LED flashlight (0.86 and 0.87) (Table 3 shown in FIG. 7).

The accuracy of correctly classified urine samples for low or high urine concentration increased incrementally, similar to the reported AUC. Starting with fluorescent light (76% for both scoring techniques), followed by halogen light (77% for over chart Uc scoring and 76% for Uc box scoring), LED light panel (79% for over chart Uc and 78% for Uc box), and ending with the highest values for the LED flashlight (79% for over chart Uc and 80% for Uc box). The rate of FP values (as a result of a low Uc, while concentration was actually above the selected USG cutoff) was inversely associated with light intensity, with the highest percentage for halogen light with the lowest light intensity (20% and 17% for the over chart and the Uc box scoring techniques, respectively) vs. LED flashlight with the highest light intensity (12% and 15% for the over chart and the Uc box scoring techniques, respectively). The ROC based Uc cut-off value for fluorescent light was one shade darker (Uc≤4) than the suggested best fit for Uc cutoff for all other light types (Uc≤3) to classify between low vs. high urine concentration.

### Difference Between Frozen and Fresh Urine Samples

Aim (c) aimed to investigate generalizability of the outcomes between fresh and frozen urine samples. The median USG for the 78 freshly measured samples was 1.018 (IQR 1.012-1.023). The median Uc score was 2, with a larger IQR in the frozen condition (+2 urine color shades) than in the freshly measured samples (P=0.004), indicating a larger number of darker scored urine samples in the frozen condition.

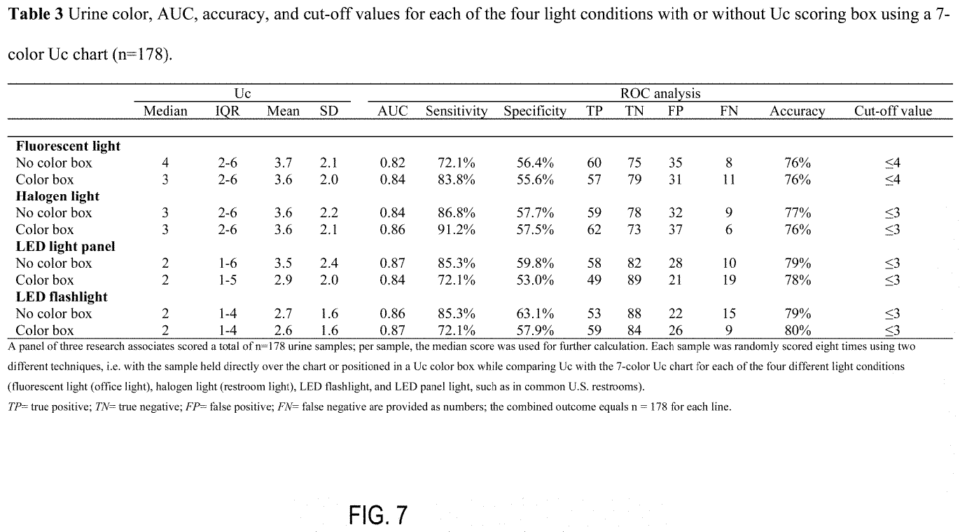

Correlations between Uc and USG were somewhat stronger for frozen samples r=0.74 (with 95% CI 0.61-0.83, P<0.001) vs. fresh samples r=0.59 (with 95% CI 0.42-0.85, P<0.001) as shown in Table 4 (shown in FIG. 8), but samples were correlated moderately for Uc when comparing fresh vs. frozen Uc scores (r=0.64 with 95% CI 0.48-0.76, P<0.001).

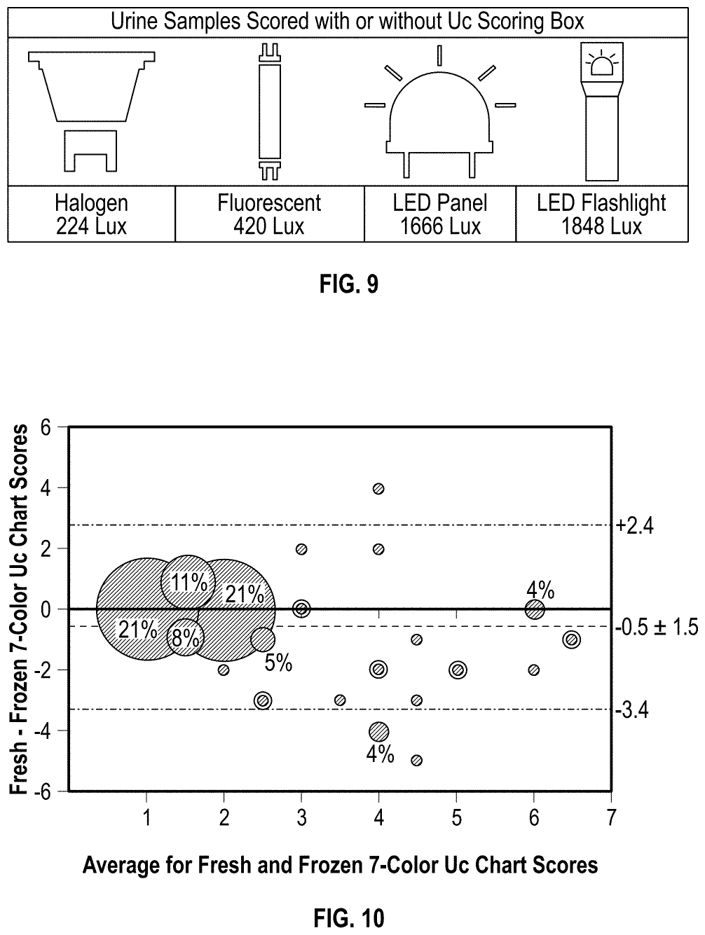

Based on the Bland-Altman plot comparing the Uc scores of frozen and fresh urine samples on an individual level, 48% of the reported Uc scores were similar for both conditions (FIGS. 10), and 75% of the reported Uc scores stayed within a ±1 Uc shade difference. When correlating the results of y-axis and x-axis of the Bland-Altman plot, a small level of reporting bias was detected (r=−0.45 (P<0.001). The nature of this bias was expressed at the mid-section of the 7-color Uc chart with slightly darker Uc scores (˜13%) in the frozen condition, showing a higher level of under-reporting in comparison to freshly scored samples. With respect to FIG. 10, the size of the bubbles indicates the percentage of urine color scores, small black dots represent 1% values, and circled dots represent 2-3% values. The average reporting bias is indicated by the dotted line denoted +0.5±1.5, and the 95% limits of agreement are indicated by the dotted lines denoted +2.4 and −3.4.

The AUC was higher for frozen samples (0.88) than for fresh samples (0.77). This resulted in lower accuracy of correctly classifying fresh samples (64.4%) in comparison to frozen samples (83.6%). The ROC based Uc cut-off value was one shade darker (Uc≤2) than the suggested best fit for Uc cutoff for freshly measured urine samples (cut-off: 1) used to classify between low vs. high urine concentration.

### Discussion

One insight of this study is that brighter LED light conditions reported a higher diagnostic ability to discriminate low vs. high urine concentration urine samples in comparison to the halogen and fluorescent light conditions. Light intensity influenced perceived Uc, but despite significant differences for Uc between all light conditions, the one shade Uc cut-off value difference between the bright (1660-1848 lux) and darker (224-420 lux) light conditions is the most important practical finding of this study. When ranking the accuracy of correctly classified urine samples to identify low vs. high urine concentration, fluorescent light (420 lux) was ranked with the lowest accuracy and the darkest optimal Uc cut-off value (≤4), while having a substantially higher light intensity than the halogen light (224 lux). Despite its lower light intensity, halogen light reported a similar Uc cut-off value ≤3 as the much brighter LED light conditions, therefore, factors other than light intensity may influence how urine color is perceived.

Regarding the results for objective (a), aiming to evaluate the scored Uc using different lights and scoring techniques, the median scores ranged from 2-4, with the lowest and highest IQR values (1 and 6, respectively) fit well within earlier reported data. Previous studies reported an average Uc of 3±1 22 and 3±2 up to 6±1 21. Earlier studies reporting Uc scoring validation reported the urine samples in a well-lit room. The question is which of the conditions matched the definition of a “well-lit room,” as light intensity varied substantially between the fluorescent, halogen, and the LED lights. This research suggests that future studies investigating Uc scoring should specify light type, as wavelength of the light type may influence how urine color is perceived and intensity, as both may influence Uc scoring accuracy. Despite the significant difference between scoring techniques under fluorescent and LED light conditions, no clear differences were seen for reported Uc or the diagnostic ability of the method suggesting that more rigid scoring conditions, such as using a separate Uc scoring box, do not necessarily result in a better performance than the traditional sample over chart method.

Objective (b) aimed to investigate the diagnostic ability of the eight different measurement conditions. This study shows a fair to good diagnostic ability based on an overall high AUC ranging from 0.77 to 0.87. These values are comparable to the AUC values ranging from 0.73-0.82 reported by athletes, but investigator scores have also been reported to be similar or higher, ranging from 0.85-0.92 24. On average, the two brighter light conditions resulted in at least one shade lower Uc cut-off value, compared to standard fluorescent and halogen light that can be found in traditional office and restroom spaces. The LED flashlight conditions also resulted in a 3-4% better accuracy for classifying low vs. high urine concentration. In addition to this relatively modest difference in accuracy, the bright LED light conditions reported a substantially lower number of FP classified urine samples. When aiming to detect high urine concentration, a substantial ˜5% fewer FP cases in the brighter light conditions is of importance as false positive values, representing a low Uc while the actual urine concentration was above the selected 1.020 USG value, will not prompt an individual to change fluid intake, which misses the sole purpose of the hydration assessment in the first place.

Interestingly, the ROC based best fit Uc cut-off value was similar for halogen and the two LED conditions, while fluorescent light (with a higher light intensity than halogen light) reported one shade darker optimal Uc cut-off value. The visual spectrum of light ranges from 360-830 nm. Light types overlap in color range but differ in color tone. For example, LED light covers a blueish range of 395-530 nm, and fluorescent light a green to yellow range of 480-570 nm, while halogen covers a range of 650-950 nm starting in the more reddish and eventually exceeding human perception. Although the study was not targeted to differentiate between different intensities within light conditions, it seems likely that wavelength can influence how Uc is perceived, in addition to light intensity. This is of importance because analysis using spectrophotometry also showed that, while urine color tends to color darker with increasing concentration, individual values of tristimulus colorimetry (CIE L_a_b_) scores changed, reporting a notable polynomial trend in color along the green-red axis, indicating a green hue in slightly dehydrated urine. Therefore, it should be taken into account that while urine color tends to increase with concentration, subtle differences in how the color is constructed in combination with the selected light condition, may influence how the urine is precepted by the human eye.

The goal of aim (c), comparing the results of frozen vs. originally scored fresh outcomes, was to generalize the results of this study to freshly scored urine samples. It is known that freezing tends to result in 0.5-0.6 (with 95% CI ranging between 0.3-0.8) darker Uc color, similar to what was reported in this study. Despite the mean difference in Uc between fresh and frozen urine samples, the correlation between Uc scores was fair, and the reporting bias based on the Bland-Altman plot was especially caused by Uc samples that would likely have been classified above the suggested Uc cut-off value to assess underhydration for urine samples in both fresh and frozen conditions. Therefore, the actual result for classifying urine samples for low vs. high urine concentration would have been the same for fresh and frozen samples. This suggests that the results of this study for accuracy of Uc scoring, apart from the actual Uc cut-off value, could be generalized to freshly measured urine samples.

This study's strengths were the relatively large sample size and that samples were frequently rotated to assure technicians did not become familiar with samples, ensuring objective Uc scoring using relevant light conditions. The outcome of this study suggests that very bright LED light conditions report the highest accuracy with the lowest number of FP misclassifications. The LED flashlight condition could be easily reproduced at low cost with only slight modifications of the light, using white masking tape or “painter's tape,” making the method accessible for a large population. The study was not without limitations. The sensitivity analysis could be based on only one light condition (LED panel) using the Uc scoring box technique, because this was the only way the color of fresh urine samples was evaluated before freezing. Another limitation is that the study used randomly selected spot urine samples provided at different times of the day, which could have influenced urine concentration. Similarly, Uc and urine concentration could have been influenced by acute rehydration, leading to a mismatch between Uc and urine concentration due to acute dilution of the urine.

In conclusion, the accuracy of Uc scoring is affected by light conditions. The most practical outcome is that the Uc cut-off value may differ between light conditions, while the scoring techniques evaluated in this study do not seem to lead to practical differences in Uc scoring accuracy. The lower light intensity conditions, i.e., halogen and fluorescent light, resulted in a 1-2 shade darker Uc and a lower Uc scoring accuracy in comparison to the much brighter LED light conditions. The suggested Uc cut-off based on the 7-color Uc chart to detect underhydration was ≤4 for fluorescent light and ≤3 for halogen and LED light conditions. The results of this study were based on previously frozen urine samples. The sensitivity analysis suggests that results can be extrapolated by subtracting one Uc shade for each light condition when determining the Uc cut-off of fresh urine samples.

Although the disclosure has been described with reference to certain preferred aspects, variations and modifications exist within the scope and spirit of one or more independent aspects of the disclosure as described. Various features and advantages of the disclosure are set forth in the following claims.

What is claimed is:

1. A urine color and concentration assessment system comprising:
a housing;
a color chart supported by the housing; and
a urine collection container supported by the housing and positioned adjacent the color chart, the urine collection container configured to receive a urine sample for comparing a color of the urine sample to a color of the color chart.

2. The urine color and concentration assessment system of claim 1, further comprising one or more light sources positioned to illuminate the color chart, a urine sample contained within the urine collection container, or both.

3. The urine color and concentration assessment system of claim 1, wherein the urine collection container is defined, at least in part, by a transparent material, and wherein the housing is configured to allow the user to view the urine sample from at least three sides.

4. The urine color and concentration assessment system of claim 1, wherein the color chart includes
a first color corresponding to a first range of urine specific gravity values;
a second color corresponding to a second range of urine specific gravity values; and
a third color corresponding to a third range of urine specific gravity values.

5. The urine color and concentration assessment system of claim 1, wherein the housing includes a recess that is coincident with an aperture along an axis and an opening positioned between a portion of the housing that defines the recess and a portion of the housing that defines the aperture, the urine collection container being removably received within the aperture and the recess, and wherein the color chart is supported by the housing adjacent to the opening.

6. The urine color and concentration assessment system of claim 5,
wherein a height of the opening is substantially the same as a height of color chart, and wherein the color chart includes three colors that increase from a lightest color to darkest color along a height of the color chart, each having a height that is one third the height of the color chart, and
wherein the lightest color corresponds to a first range of urine specific gravity values that include values that are less than or equal to 1.017, the darkest color corresponds to a first range of urine specific gravity values that include values that are greater than or equal to 1.022, and wherein an intermediate color between the lightest color and the darkest color corresponds to a third range of values that are between 1.017 and 1.022.

7. The urine color and concentration assessment system of claim 1, wherein the housing includes a first bore that has a first axis, a first window or opening positioned in a wall of the first bore, a second bore that defines a second axis, and a second window or opening positioned in a wall of the second bore, the first bore being positioned adjacent to the second bore and the first axis and the second axis being parallel to one another, the first bore configured to removably receive the color chart such that the color chart is viewable through the first window or opening and the second bore configured to removably receive the urine collection container such that the urine collection container is viewable through the second window or opening.

8. The urine color and concentration assessment system of claim 7,
wherein an LED is positioned within the housing adjacent to one or both of the color chart and the urine collection container to illuminate one or both of the color chart and the urine collection container;
wherein the color chart includes three colors that increase from a lightest color to darkest color along a height of the color chart, each having a height that is one third the height of the color chart, and
wherein the lightest color corresponds to a first range of urine specific gravity values that include values that are less than or equal to 1.017, the darkest color corresponds to a first range of urine specific gravity values that include values that are greater than or equal to 1.022, and wherein an intermediate color between the lightest color and the darkest color corresponds to a third range of values that are between 1.017 and 1.022.

9. A urine color and concentration assessment system comprising:
a urine collection container configured to receive a void of undiluted urine;
a color chart positioned adjacent to the urine collection container for determining hydration status from the void of undiluted urine; and
a light source positioned adjacent to one or both of the color chart and the container to illuminate one or both of the color chart and the container.

10. The urine color and concentration assessment system of claim 9, wherein the light source includes an LED.

11. The urine color and concentration assessment system of claim 10, wherein the color chart includes
a first color corresponding to a first range of urine specific gravity values;
a second color corresponding to a second range of urine specific gravity values; and
a third color corresponding to a third range of urine specific gravity values.

12. The urine color and concentration assessment system of claim 11, wherein the first range of urine specific gravity values includes values that are less than 1.017, the third range of urine specific gravity values includes values that are greater than 1.022, and the second range of urine specific gravity values include values between the first and the third ranges of urine specific gravity values.

13. The urine color and concentration assessment system of claim 11, wherein the second color is darker than the first color and the third color is darker than the second color.

14. The urine color and concentration assessment system of claim 10, wherein the light source is positioned behind the color chart.

15. The urine color and concentration assessment system of claim 10, wherein the light source is positioned behind the container.

16. The urine color and concentration assessment system of claim 10, wherein the light source is a first light source is positioned behind the color chart, and further comprising a light source is positioned behind the container.

17. A color chart for determining hydration status from a void of undiluted urine, the color chart consisting of seven colors, each of the seven colors corresponding to a range of urine specific gravity values.

18. The color chart of claim 17, wherein a first of the seven colors is the lightest color of the seven colors and corresponds to a range of specific gravity values that includes values that are less than 1.017 and a second of the seven colors is the darkest of the seven colors and corresponds to a range of specific gravity values that includes values that are greater than 1.031.

19. The color chart of claim 18, wherein a third color, a fourth color, a fifth color, a sixth color, and a seventh color are darker than the first color and lighter than the second color.

20. The color chart of claim 18, wherein a third color, a fourth color, a fifth color, a sixth color, and a seventh color correspond to ranges of specific gravity values between 1.017 and 1.031.
